(19) United States
(12) Patent Application Publication
(10) Pub. No.: US 2010/0076332 A1
(43) Pub. Date: Mar. 25, 2010

(54) EXPIRATORY FLOW SENSOR SYSTEM AND METHOD

(75) Inventors: Craig Robert Tolmie, Stoughton, WI (US); Timothy Patrick McCormick, Fitchburg, WI (US)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, NY (US)

(21) Appl. No.: 12/236,301

(22) Filed: Sep. 23, 2008

(51) Int. Cl. A61B 5/087; A61M 16/00

(52) U.S. Cl. 600/538; 128/200.24

(57) ABSTRACT

An expiratory flow sensor system is disclosed herein. The expiratory flow sensor includes an expiratory channel adapted to transfer an expiratory gas, a fresh gas channel adapted to transfer a fresh gas, and a sensing element disposed within the fresh gas channel so that the sensing element is never directly exposed to the expiratory gas. The sensing element is configured to measure a fresh gas flow rate. The fresh gas flow rate may be implemented to estimate an expiratory flow rate in a manner that minimizes imprecision attributable to sensing element contamination.

### FIELD OF THE INVENTION

This disclosure relates generally to an expiratory flow sensor system and method for a medical ventilator.

### BACKGROUND OF THE INVENTION

Medical ventilators are used to provide respiratory support to patients undergoing anesthesia and respiratory treatment whenever the patient's ability to breath is compromised. The primary function of the medical ventilator is to maintain suitable pressure and flow of gases inspired and expired by the patient. Gas flow may, for example, be maintained based on feedback from an expiratory flow sensor. The gases measured by the expiratory flow sensor can contain contaminants such as water vapor, mucus, arasolized drugs, etc. One problem is that the contaminants can interfere with the sensing element thereby reducing the precision with which expiratory flow is estimated and/or the reliability of the sensing element.

### BRIEF DESCRIPTION OF THE INVENTION

The above-mentioned shortcomings, disadvantages and problems are addressed herein which will be understood by reading and understanding the following specification.

In an embodiment, an expiratory flow sensor system includes an expiratory channel adapted to transfer an expiratory gas, a fresh gas channel adapted to transfer a fresh gas, and a sensing element disposed within the fresh gas channel so that the sensing element is never directly exposed to the expiratory gas. The sensing element is configured to measure a fresh gas flow rate. The fresh gas flow rate may be implemented to estimate an expiratory flow rate in a manner that minimizes imprecision attributable to sensing element contamination.

In another embodiment, an expiratory flow sensor system includes an expiratory channel adapted to transfer an expiratory gas, and a flow restrictor disposed within the expiratory channel. The expiratory flow sensor system also includes a fresh gas channel adapted to transfer a fresh gas, and a sensing element disposed within the fresh gas channel such that the sensing element is never directly exposed to the expiratory gas. The sensing element is configured to measure a fresh gas flow rate. The expiratory flow sensor system also includes a controller operatively connected to the sensor. The controller is configured to estimate an expiatory flow rate based on the fresh gas flow rate.

In another embodiment, a method includes providing an expiratory channel, providing a fresh gas channel pneumatically coupled with the expiratory channel, and providing a sensing element disposed within the fresh gas channel. The method also includes transferring a fresh gas through the fresh gas channel, implementing the sensing element to measure a fresh gas flow rate, and estimating an expiratory gas flow rate based on the fresh gas flow rate.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram illustrating a ventilator system connected to a patient in accordance with an embodiment;

FIG. 2 is a detailed schematic illustration of an expiratory flow sensor in accordance with an embodiment; and

FIG. 3 is a detailed schematic illustration of an expiratory flow sensor in accordance with another embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

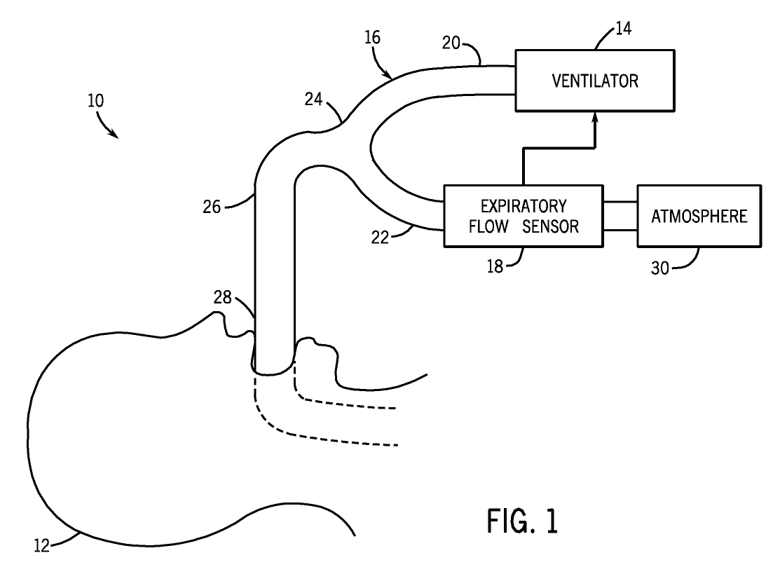

Referring to FIG. 1, a schematically illustrated ventilator system 10 is shown connected to a patient 12 in accordance with an exemplary embodiment. The ventilator system 10 includes a ventilator 14, a breathing circuit 16, and an expiratory flow sensor 18.

The ventilator 14 provides breathing gasses to the patient 12 via the breathing circuit 16. The ventilator 14 regulates the volume of gasses transferred into the breathing circuit 16 and/or the pressure level of the gasses within the breathing circuit 16 based in part on feedback from the expiratory flow sensor 18.

The breathing circuit 16 includes an inspiratory branch 20, an expiratory branch 22, a Y-connector 24, a patient branch 26, and an interface 28. The interface 28 is the portion of the breathing circuit 16 that is directly coupled with the patient 12. According to the embodiment depicted, the interface 28 comprises an endotracheal tube, however it should be appreciated that other known devices may also be implemented for the interface 28.

According to one embodiment, breathing gasses are transferred from the ventilator 14, through the inspiratory branch 20, the Y-connector 24, the patient branch 26, the interface 28, and are then delivered into the patient's lungs (not shown). After the breathing gasses are delivered into the patient's lungs, the patient 12 passively exhales due to the elasticity of his or her lungs. The exhaled gas from the patient's lungs is transferred through the interface 28, the patient branch 26, the Y-connector 24, the expiratory branch 22, the expiratory flow sensor 18, and is then vented to atmosphere 30 or a collection system (not shown). Before the exhaled gas is vented to atmosphere 30, the expiratory flow sensor 18 estimates one or more characteristics of the expiratory gas flow. Data corresponding to the estimated expiratory flow characteristics is transmitted from the expiratory flow sensor 18 to the ventilator 14. The ventilator 14 may, for example, implement this data to regulate the volume of breathing gasses delivered during a subsequent breathing cycle or report an independent exhaled volume measurement.

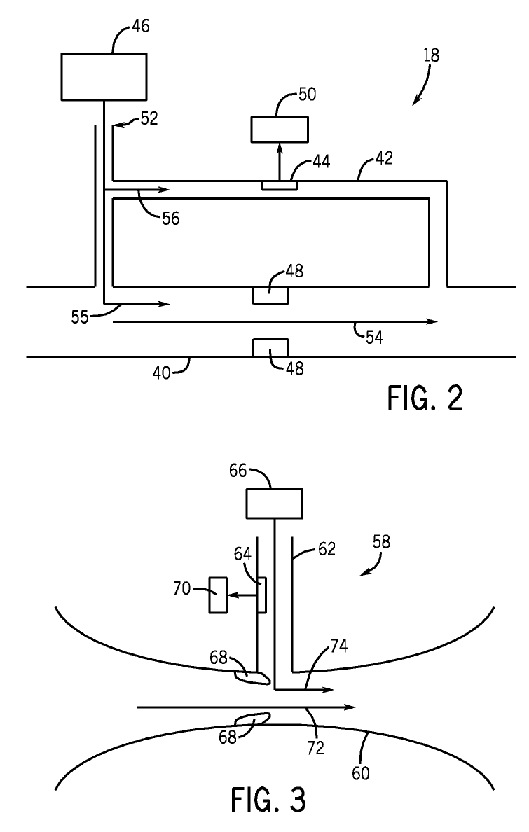

Referring to FIG. 2, the expiratory flow sensor 18 is shown in accordance with an embodiment. The expiratory flow sensor 18 includes an expiratory channel 40, a fresh gas channel 42, a sensing element 44, a source of fresh gas 46, and a flow restrictor 48. According to one embodiment, the expiratory flow sensor 18 also includes a controller 50 operatively connected to the sensing element 44. It should, however, be appreciated that the controller 50 may alternatively be remotely located and incorporated, for example, as a component of the ventilator 14 (shown in FIG. 1).

The source of fresh gas 46 may, for example, comprise a pressurized storage tank or pump adapted to deliver a generally contaminant free gas. For purposes of this disclosure, a contaminant free gas is one that does not include any of the contaminants commonly associated with ventilation expiration such as mucus, arasolized drugs, etc.

The expiratory channel 40 is pneumatically coupled in parallel with the fresh gas channel 42. The diameter of the expiratory channel 40 is generally larger than that of the fresh gas channel 42 in order to allow for a higher flow rate, however the respective diameters may be configurable to meet the needs of a particular application. The fresh gas channel 42 includes a fresh gas inlet 52 coupled with the source of fresh gas 46. The flow restrictor 48 is disposed within the expiratory channel 40, and the sensing element 44 is disposed within the fresh gas channel 42. The expiratory channel 40 is adapted to accommodate an expiratory flow represented by the arrow 54 and/or a fresh gas flow represented by the arrow 55. The fresh gas channel 42 is exclusively adapted to accommodate a fresh gas flow represented by the arrow 56.

The flow restrictor 48 forms a constriction reducing the effective inner diameter of the expiratory channel 40. Fluid flow through the constriction generates a pressure differential across the flow restrictor 48. The pressure differential would tend to divert at least a portion of any expiratory gasses from the patient 12 (shown in FIG. 1) into the fresh gas channel 42 in the absence of the fresh gas flow described in detail hereinafter.

The source of fresh gas 46 is configured to introduce fresh gas into the fresh gas inlet 52 at a rate sufficient to prevent expiratory gasses from entering the fresh gas channel 42. The fresh gas introduced into the fresh gas inlet 52 takes the path of least resistance through one or both of the fresh gas channel 42 and the expiratory channel 40. As an example, if no expiratory gas is passing through the expiratory channel 40, half of the fresh gas may pass through the fresh gas channel 42 and the other half of the fresh gas may pass through the expiratory channel 40. Similarly, if a high volume of expiratory gas is passing through the expiratory channel 40, all the fresh gas introduced into the fresh gas inlet may be passed through the fresh gas channel 42.

Implementing fresh gas to prevent expiratory gasses from entering the fresh gas channel 42 in the manner described hereinabove has the effect of protecting the sensing element from expiratory contaminants. More precisely, by preventing the expiratory gasses from entering the fresh gas channel 42 in which the sensing element 44 is disposed, the sensing element 44 is never directly exposed to the expiratory gasses or any constituent contaminants. As the sensing element 44 only comes into contact with contaminant free fresh gas and is never exposed to expiratory gasses, the precision and reliability of the sensing element 44 cannot become diminished as a result of expiratory contaminant exposure.

During operation, the sensing element 44 can be configured to measure the flow rate of the fresh gas passing through the fresh gas channel 42. Thereafter, the controller 50 can estimate the flow rate of the expiratory gas from the patient 12 (shown in FIG. 1) based on the fresh gas flow rate measurements. According to one embodiment, this conversion is accomplished by calibrating expiratory flow sensor 18 relative to a conventional flow sensor. According to another embodiment, the difference between the volume of fresh gas introduced into the fresh gas inlet 52 and the volume of fresh gas measured at the sensing element 44 may be implemented to estimate expiratory gas flow rate.

Referring to FIG. 3, an expiratory flow sensor 58 representing an alternate embodiment of the expiratory flow sensor 18 (shown in FIG. 2) is shown in detail. The expiratory flow sensor 58 includes an expiratory channel 60, a fresh gas channel 62, a sensing element 64, a source of fresh gas 66, and a flow restrictor 68. According to one embodiment, the expiratory flow sensor 58 also includes a controller 70 operatively connected to the sensing element 64. It should, however, be appreciated that the controller 70 may alternatively be remotely located and incorporated, for example, as a component of the ventilator 14 (shown in FIG. 1).

The fresh gas channel 62 is pneumatically coupled with the expiratory channel 60 at a downstream position as measured relative to the flow restrictor 68. The diameter of the expiratory channel 60 is generally larger than that of the fresh gas channel 62 in order to allow for a higher flow rate, however the respective diameters may be configurable to meet the needs of a particular application. The fresh gas channel 62 is pneumatically coupled with the source of fresh gas 66 that may, according to one embodiment, comprise atmospheric air. The flow restrictor 68 is disposed within the expiratory channel 60, and the sensing element 64 is disposed within the fresh gas channel 62. The expiratory channel 60 is adapted to accommodate an expiratory flow represented by the arrow 72 and/or a fresh gas flow represented by the arrow 74. The fresh gas channel 62 is exclusively adapted to accommodate the fresh gas flow represented by the arrow 74.

The flow restrictor 68 forms a constriction reducing the effective inner diameter of the expiratory channel 60. Fluid flow through the constriction generates a low-pressure region downstream from the flow restrictor 68 in accordance with the Venturi effect, which is well known to those skilled in the art. This low-pressure region pulls fresh gas from the source of fresh gas 66. It should be appreciated that the sensing element 64 disposed within the fresh gas channel only comes into contact with fresh gas drawn from the source of fresh gas 66. As the sensing element 64 is never exposed to expiratory gasses, the precision and reliability of the sensing element 64 cannot become diminished as a result of expiratory contaminant exposure.

During operation, the sensing element 64 can be configured to measure the flow rate of the fresh gas passing through the fresh gas channel 62. Thereafter, the controller 70 can estimate the flow rate of the expiratory gas from the patient 12 (shown in FIG. 1) based on the fresh gas flow rate measurements. According to one embodiment, this conversion is accomplished by calibrating expiratory flow sensor 58 relative to a conventional flow sensor.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. An expiratory flow sensor system comprising:
an expiratory channel, said expiratory channel adapted to transfer an expiratory gas;
a fresh gas channel pneumatically coupled with the expiratory channel, said fresh gas channel adapted to transfer a fresh gas; and
a sensing element disposed within the fresh gas channel such that the sensing element is never directly exposed to the expiratory gas, said sensing element being configured to measure a fresh gas flow rate;
wherein the fresh gas flow rate may be implemented to estimate an expiratory flow rate in a manner that minimizes imprecision attributable to sensing element contamination.

2. The expiratory flow sensor system of claim 1, further comprising a controller operatively connected to the sensing element, said controller being configured to estimate an expiatory flow rate based on the fresh gas flow rate.

3. The expiratory flow sensor system of claim 2, further comprising a flow restrictor disposed within the expiratory channel.

4. The expiratory flow sensor system of claim 3, wherein the fresh gas channel is pneumatically coupled to the expiratory channel at a downstream location as measured relative to the flow restrictor.

5. The expiratory flow sensor system of claim 4, wherein the flow restrictor is configured to generate a low-pressure region adapted to pull fresh gas into the fresh gas channel.

6. The expiratory flow sensor system of claim 3, wherein the fresh gas channel is pneumatically coupled in parallel with the expiratory channel.

7. The expiratory flow sensor system of claim 6, further comprising a source of fresh gas connected to the fresh gas channel.

8. The expiratory flow sensor system of claim 7, wherein the source of fresh gas comprises one of a pressurized storage tank and a pump.

9. The expiratory flow sensor system of claim 7, wherein the source of fresh gas is configured to introduce fresh gas into the fresh gas channel at a rate sufficient to prevent any expiratory gas form entering the fresh gas channel.

10. An expiratory flow sensor system comprising:
an expiratory channel, said expiratory channel adapted to transfer an expiratory gas;
a flow restrictor disposed within the expiratory channel;
a fresh gas channel pneumatically coupled with the expiratory channel, said fresh gas channel adapted to transfer a fresh gas;
a sensing element disposed within the fresh gas channel such that the sensing element is never directly exposed to the expiratory gas, said sensing element being configured to measure a fresh gas flow rate; and
a controller operatively connected to the sensing element, said controller being configured to estimate an expiatory flow rate based on the fresh gas flow rate.

11. The expiratory flow sensor system of claim 10, wherein the fresh gas channel is pneumatically coupled to the expiratory channel at a downstream location as measured relative to the flow restrictor.

12. The expiratory flow sensor system of claim 11, wherein the flow restrictor is configured to generate a low-pressure region adapted to pull fresh gas into the fresh gas channel.

13. The expiratory flow sensor system of claim 10, wherein the fresh gas channel is pneumatically coupled in parallel with the expiratory channel.

14. The expiratory flow sensor system of claim 13, further comprising a source of fresh gas connected to the fresh gas channel.

15. The expiratory flow sensor system of claim 14, wherein the source of fresh gas is configured to introduce fresh gas into the fresh gas channel at a rate sufficient to prevent any expiratory gas form entering the fresh gas channel.

16. A method comprising:
providing an expiratory channel;
providing a fresh gas channel pneumatically coupled with the expiratory channel;
providing a sensing element disposed within the fresh gas channel;
transferring a fresh gas through the fresh gas channel;
implementing the sensing element to measure a fresh gas flow rate; and
estimating an expiratory gas flow rate based on the fresh gas flow rate.

17. The method system of claim 16, wherein said transferring a fresh gas through the fresh gas channel comprises implementing a flow restrictor to generate a low-pressure region.

18. The method system of claim 16, wherein said transferring a fresh gas through the fresh gas channel comprises transferring a fresh gas through the fresh gas channel at a rate sufficient to prevent any expiratory gas form entering the fresh gas channel.
